(19) United States
(12) Patent Application Publication
(10) Pub. No.: US 2026/0055364 A1
(43) Pub. Date: Feb. 26, 2026

(54) Methods for Improving Sperm Function and Fertilizing Ability for Assisted Reproduction Techniques

(71) Applicants: Dario KRAPF, Rosario Sante Fe (AR); Consejo Nacional de Investigaciones Cientificas y Técnicas (CONICET), Ciudad Autonoma de Buenos Aires (AR); Universidad Nacional de Rosario, Rosario Sante Fe (AR)

(72) Inventors: Dario KRAPF, Rosario (AR); Mariano Gabriel BUFFONE, Caba (AR); Guillermina Maria LUQUE, Caba (AR); Matias GOMEZ ELIAS, Caba (AR)

(21) Appl. No.: 19/104,392

(22) PCT Filed: Aug. 19, 2022
(86) PCT No.: PCT/EP2022/073215

§ 371 Date: Feb. 18, 2025

(51) Int. Cl. C12N 5/076; C12N 5/00; C12N 5/073

(52) U.S. Cl. CPC C12N 5/061; C12N 5/0604; C12N 5/562; C12N 2500/14; C12N 2500/16; C12N 2500/42; C12N 2500/62

(57) ABSTRACT

The disclosure provides methods for improving sperm function and fertilizing ability for assisted reproduction techniques. The methods provided by the disclosure, in some embodiments entail sequential changes of temperature, bicarbonate concentration, albumin concentration and calcium ionophore.

### BACKGROUND

Male factor is a contributing factor for ˜50% of couples having difficulty conceiving. An important aspect of assisted reproduction is obtaining optimal function of male gametes (sperm) to help maximize the fertilization process and the availability of embryos for transfer. Accordingly, a need exists for media, compositions, and methods for increasing sperm function, although several approaches have been tried as detailed below:
A. Procedures that isolate the best sperm sub-population of a sample. This procedure intends to enrich a sample with competent cells. Most of these widely used methods select sperm based on their motility. Examples of these methods are discontinuous gradient centrifugation, swim-up, or the use of microfluidic devices. They are widely accepted in the assisted reproductive techniques (ART) clinics. More recently, a report by Serafin Pérez-Cerezales et al (2018) indicated that a high-quality sperm subpopulation was in vitro selected by thermotaxis and that this subpopulation is the one that showed much higher DNA integrity and lower chromatin compaction compared to raw sperm sample. In mice, these sperm gave rise to more and better embryos through intracytoplasmic sperm injection (ICSI), doubling the number of successful pregnancies. ICSI procedures face the problem of sperm selection among a highly heterogenous sample. Thus, these procedures that enrich a population with fertilizing competent sperm would frequently give better results. However, these procedures do no enhance sperm function, and only isolate those sperm with higher fertilizing competence.
B. Procedures that intended to predict fertilization outcome.
   1. The work by Alvarez et al (1996) reported a procedure where human sperm were subjected to heat stress to see whether motility was enhanced. The result of this procedure was later correlated to the in vitro fertilization (IVF) outcome performed with a second semen sample obtained from the same patient. This procedure adds a predictive tool to the clinic, where they show that those patients whose sperm samples increase their motility upon heat stress have better fertilization chances.
   2. The work by Mann et al (2002) intended to correlate DNA integrity after heat induced stress with sperm capacitation index. Authors indicated that heat induced damage correlated with poor sperm characteristics.
C. Procedures that induce the sperm asymmetric motility type of movement named hyperactivation (HA), through increased temperature incubation.
   1. Si et al (1997). In this study, the authors investigated the response of hamster sperm to temperature incubations ranging from 22° C. to 40° C. during 3.5 h, to observe a temperature-dependent HA, whereas higher temperatures yielded higher HA. This type of long stress heat usually promotes DNA damage derived from reactive oxygen species.
   2. Keepler et al (1999). In this report, the authors showed that when a cryopreserved sperm straw is incubated simultaneously by halves, one at 40° C. and the other at 37° C., the higher temperature half accumulates more sperm, displaying higher HA values. These results are consistent with higher HA induced by 40° C. and thermotaxis effect of increasing temperature from 37° C. to 40° C.
   3. Chan et al (1998). These authors reported that incubation of human sperm at 40° C. for up to 4 h improved sperm HA and gave raise to higher penetration of zona-free eggs, as a test to evaluate a sperm fertilizing ability.
   4. Küçük et al (2008). In this report, the authors showed that incubating human sperm for 2 h at 40° C. yielded higher percentages of motile sperm with higher pregnancy rates after intrauterine insemination (IUI), compared to sperm incubated at 37° C.
   5. Chan et al (1998)). In this study, the authors reported that higher HA at 40° C. correlates with successful IUI when those samples were processed with standard procedures at standard temperature (7 pregnant women out of 44 patients). This procedure is proposed as a test to predict IUI success.
D. Procedures using calcium (Ca²⁺) ionophores to enhance sperm motility and function.
   1. Tateno et al (2012). In this article, the authors demonstrated that the application of high concentrations (10-20 μM) of the Ca²⁺ ionophore A23187 for 10 min immobilizes mouse sperm. After removal of the ionophore, mouse sperm acquires HA. Worth noticing, human sperm does not regain motility after ionophore treatment, in a clear difference among species. Furthermore, Sanchez Cardenas et al (2018) showed that while 5-10 μM A23187 substantially elevated intracellular Ca²⁺ concentrations and immobilized sperm in a few minutes, smaller concentrations (0.5 and 1 μM) provoked sperm HA without immobilization. This is in line with previous knowledge indicating that intracellular Ca²⁺ concentration thresholds switch motility on and off.
   2. Navarrete et al (2016). In this article, the authors showed that treatment of mouse sperm with high concentrations (10-20 μM) of the Ca²⁺ ionophore A23187 for 10 min with posterior removal overcomes infertility observed in sperm from CatSper1^{-/-}, Slo3^{-/-} and Adcy10^{-/-} knock-out (KO) lines.
E. Other methods to improve sperm function.
   1. Navarrete et al (2019) showed that upon energy restriction in the incubation media, mouse sperm ceased moving. When energy substrates were added back to the media, sperm motility was rescued. Following rescue, a significantly higher percentage of starved sperm attained hyperactivated motility and displayed increased ability to fertilize in vitro when compared with sperm persistently incubated in standard capacitation media. This technology (WO2017173391A1) has proven ineffective for the induction of capacitation related events in human sperm, as further showed by Marin Briggiler et al (2021).

The method herein described is based on proper manipulation of both temperature incubation and intracellular sperm Ca²⁺ to develop a synergistic effect HA and blastocysts formation rates. HA is a key parameter when analyzing the outcome of IVF (Wiser et al, 2014). It has been shown that among patients with increased HA, 93.3% had high fertilization rates compared to 64% found in the group with no increase of HA. More importantly, this method improves developmental rates of fertilized eggs up to the state of blastocysts and provides higher blastocysts scores, which directly correlates to better pregnancy rates.

### BRIEF DESCRIPTION OF THE INVENTION

i) In some embodiments, provided herein is a method of inducing increased sperm function comprising; (a) incubating a mammalian sperm in specific conditions of temperature, bicarbonate (HCO₃) concentration, albumin concentration and Ca²⁺ ionophores, thereby inducing increased sperm function compared to a suitable control sperm. (b) The mammalian sperm provided by (a) is suitable for use in the ART.

ii) In some embodiments, the method is performed in vitro. In some embodiments of the method, step (b) is performed in vivo, in the reproductive tract of a female subject, by IUI of the mammalian sperm from step (a).

iii) In some embodiments, the increased sperm function comprises an increase in motility as measured by computer assisted semen analysis (CASA). In some embodiments, the increase in motility comprises an increase in curvilinear velocity (VCL) of the mammalian sperm as well as in percentage of hyperactivated sperm. In some embodiments, the increased sperm function comprises an increase in mammalian sperm capacitation as measured by an increase in the ability to fertilize an egg. In some embodiments, the increased sperm function comprises generation of an embryo with increased viability, and/or improved implantation relative to an embryo generated with a suitable control sperm, or increased ability of an embryo to develop to at least a 2-cell developmental stage, blastocyst developmental stage or an offspring relative to an embryo generated with a suitable control sperm.

iv) In some embodiments, the mammalian sperm is a human, non-human primate, porcine, bovine, equine, ovine, canine, feline, or murine sperm. In some embodiments, the mammalian sperm is a human sperm from a normospermic male, a subfertile male, oligospermic male, a teratospermic male, or an asthenospermic male.

v) In some embodiments, the one or more sperm function is selected from VCL, amplitude of lateral head displacement (ALH), sperm capacitation, percentage of hyperactivated sperm. In some embodiments, the albumin is of human, bovine or from other species origin or, a synthetic serum substitute.

vi) In some embodiments, the temperature used following ionophore incubation can be adjusted according to the species from 37.5° C. to 45° C., preferentially from 39° C. to 44° C., more precisely from 40° C. to 43° C.

vii) In some embodiments, the Ca²⁺ ionophore of use can be different according to the species, including (but not restricted to) 4-Bromo-A23187 (4-Br-A23187), A23187, ionomycin, Ca²⁺ ionophore I-V, including any means to pharmacologically increase the intracellular Ca²⁺ concentration temporarily.

### BRIEF DESCRIPTION OF DRAWINGS

FIGS. 1A-D depict how changes of incubation temperature and intracellular Ca²⁺ concentration in human sperm produce a synergistic effect in sperm kinematic parameters. In the Control treatment, sperm were incubated for 2 min in the presence of 0.2% DMSO, and for additional 30 min at 37° C. In the Temperature treatment, sperm were incubated for 2 min in the presence of 0.2% DMSO, followed by 30 min at 40° C. In the Ionophore treatment, sperm were incubated for 2 min in the presence of 5 μM 4-Br-A23187 (0.2% DMSO), followed by 30 min at 37° C. In the Combined treatment, sperm were incubated for 2 min in the presence of 5 μM 4-Br-A23187, followed by 30 min at 40° C. Total motility (FIG. 1A), amplitude of lateral head movement (ALH) (FIG. 1B), curvilinear velocity (VCL) (FIG. 1C) and hyperactivation (HA) (FIG. 1D) were recorded. Data are presented as mean±standard error of the mean (SEM), n=7. Statistical significance was analyzed with analysis of variance (ANOVA) and Tukey post-test. Different letters indicate statistically significant differences, p<0.05.

FIGS. 2A-B show the effect of different concentration and/or different Ca²⁺ ionophores on sperm hyperactivation (HA). In FIG. 2A, sperm were incubated with either 0.2% DMSO for 2 min, 5 μM 4-Br-A23187 for 2 min, 1 μM 4-Br-A23187 for 5 min or 0.5 μM A23187 for 2 min, and later exposed to 40° C. for 30 min. Data are presented as mean±SEM of at least 3 independent experiments. Statistical significance was analyzed with ANOVA and Tukey post-test. *p<0.05. In FIG. 2B, the effect of different temperatures of incubation on sperm hyperactivation (HA) is analyzed after the Ionophore treatment. Sperm were incubated with 5 μM 4-Br-A23187 for 2 min followed by 60 min at either 37, 38, 39, 40, 41, 42 or 43° C. Data are presented as mean±SEM of at least 3 independent experiments.

FIGS. 3A-C show the effect of different incubating periods on sperm hyperactivation (HA) from three donors when incubated at either 37° C. or 40° C. over 4 h.

FIGS. 4A-B illustrates how the combined effect of temperature and Ca²⁺ ionophore treatment in sperm produces a higher number of human blastocysts compared to conventional IVF. Sperm from Control and Combined treatments were used in clinical assisted reproduction IVF procedures. The number of fertilized eggs (two pronuclei at day 2) and the number of high-quality blastocysts at day 5 and 6 were recorded. The percentage of blastocyst per mature egg (FIG. 4A) and per fertilized egg (FIG. 4B) were determined. Statistical significance was analyzed with Fisher's exact test, *p<0.05.

### DETAILED DESCRIPTION OF INVENTION

### Definitions

### Sperm Function

i) In some embodiments, provided herein is a method for increasing sperm function. The method comprises incubating a mammalian sperm under different conditions in a sequential manner prior to inseminating or injecting an egg in assisted reproduction.

The sequential incubating procedure consist of five steps: (a) washing mammalian sperm with medium containing neither HCO₃⁻ nor albumin for 5 min and resuspended in the same washing medium; (b) the mammalian sperm provided by step (a) is incubated for 1 to 20 min, preferentially 3 to 6 min, with 0.01 to 20 μM, preferentially 0.1 to 6 μM of a Ca²⁺ ionophore, preferentially 4-Br-A23187 at 36° C. to 38° C.; (c) the mammalian sperm provided by step (b) is washed with medium containing up to 1% albumin for 1-10 min and resuspended in the same washing medium and incubated for 10-60 min (preferentially 20-40 min) at 36-38° C.; (d) the mammalian sperm provided by step (c) is incubated in a medium containing 3-9 mM HCO₃⁻, preferentially 6 mM, and 0.1-1% albumin, preferentially 0.5% albumin for 30-90 min, preferentially 60 min at 38-43° C., preferentially at 40° C. (e) the appropriate concentration of mammalian sperm provided by step (d) is then transferred to a medium containing 10-30 mM HCO₃⁻, preferentially 25 mM HCO₃ and 0.1-1% albumin, preferentially 0.5% albumin at 37° C. This sequential procedure increased sperm function compared to a suitable control sperm and are suitable for use in ART.

In some embodiments, the insemination of eggs is performed in vitro (IVF or ICSI) or in vivo, for example, by cervical or IUI of the sperm which has been previously incubated in the sequential procedure.

Increased sperm function includes one or more of the following aspects: increased motility such as the percentage of sperm in a population exhibiting hyperactivated motility as assessed by CASA, increased capacitation, and increased rates of fertilization, e.g., development to at least 2-cells, blastocyst development, or live birth. Accordingly, in some embodiments, sperm function can be sperm motility, VCL, ALH, sperm capacitation, percentage of hyperactivated sperm, ability to fertilize an egg, generation of an embryo. In some embodiments, the embryo generated by the sperm with increased function comprises one or more characteristics selected from increased viability, increased implantation, increased ability to develop to at least 2-cell developmental stage, blastocyst developmental stage or an offspring.

ii) An increase in one or more sperm functions, as contemplated herein, constitutes an increase in one or more sperm functions relative to a suitable control sperm. In some embodiments, the one or more sperm functions can be increased by at least 5%, 10%, 20%, 30%, 40%, 50%, 60%, 75%, 80%, 90%, 100%, 200%, 300% or more. In some embodiments, the one or more sperm functions can be increased by from 10% to 200%, from 25% to 150%, from 50% to 100%, or from 70% to 90%.

iii) As it relates to the present disclosure, sperm “activity” and/or “function” encompass physiological processes such as, for example, sperm motility, thermo, rheo and/or chemotaxis and the ability to fertilize an egg. The terms “activity” and/or “function” can further include processes which occur prior to, during fertilization and/or interaction with the egg surrounding layers such as capacitation and acrosome reaction (also known as acrosomal exocytosis) or fusion with the plasma membrane, and/or processes after fertilization of the egg, for example, formation of an embryo. In some embodiments, the embryo exhibits increased (longer) viability, improved implantation, and/or ability to develop to a 2-cell stage, a blastocyst, or to an offspring resulting in live birth.

### Sperm Motility

i) The term “motility” is strictly associated to the general movement of the cell, but it also may be applied to other aspects of motility such as, the speed of movement of a sperm cell and/or any increase or decrease in the proportion of moving sperm cells in any given population. As such, the methods disclosed herein may be used not only to increase sperm motility, but also to increase the speed of movement of a sperm cell and/or the proportion (percentage) of moving cells in any given population of sperm.

ii) Motility of sperm is expressed as the total percent of motile sperm, or the velocity of sperm that are motile. These measurements may be made by a variety of assays but are conveniently assayed in one of two ways. Either a subjective visual determination is made using a phase contrast microscope when the sperm are placed in a microscope slide, or a CASA is used. Under phase contrast microscopy, motile and total sperm counts are made, and speed is assessed as fast, medium or slow. A second method of assessing sperm motility is by using a CASA, the motility characteristics of individual sperm cells in a sample are objectively determined.

iii) Accordingly, the term “motility” encompasses percentage of motile sperm which can be the percentage of the total number of sperm assessed that fall within all World Health Organization (WHO) categories of motility except the category designated “no motility” regardless of velocity or directionality. Manual counting classifies sperm cells into 4 categories (immotile, locally motile, nonlinear and linear motile) using qualitative subjective criteria of selection.

iv) The term “motility” encompasses percentage of motile sperm, i.e., the percentage of total number of sperm assessed in a population exhibiting progressive motility, hyperactivated motility based on CASA.

v) The methods disclosed herein can increase percentage of progressive motility sperm, e.g., percentage of sperm exhibiting linear movement from one point to another, with turns of the head of less than 90 degrees from sperm that are otherwise non-progressive, i.e., sperm that move but do not make forward progression. In some embodiments, the increased motility comprises increase in percentage of hyperactivated sperm. Hyperactivated sperm motility is characterized by sperm that have a high amplitude, asymmetrical beating pattern of the flagellum. Hyperactivated motility is characterized by vigorous movement with many seemingly random variations without a well-defined progressive path and turns of the sperm head of greater than 90 degrees. Hyperactivated sperm motility is more vigorous and short term than progressive motility. Biologically, hyperactivated sperm motility is important to enable sperm to traverse the egg outer investments prior to fertilizing the mature egg. In some embodiments, the methods disclosed herein can increase percentages of hyperactivated sperm in a given population of sperm.

vi) It should be understood that other standardized measures of sperm motility parameters can also be used. Other measures of sperm motility include “velocity” and “linearity” which can be assessed using automatic semen analyzers. In some embodiments, the methods disclosed herein can increase sperm function comprising increase in average path velocity (VAP), straight-line velocity (VSL), VCL, ALH and beat cross frequency (BCF) or other movement parameters of the sperm including parameters known to those of skill in the art. VAP is the velocity along the average path of the sperm cell. VSL is the linear or progressive velocity of the cell. VCL is the measure of the rate of travel of the centroid of the sperm head over a given time period. Linearity of forward progression (LIN) is the ratio of VSL to VCL and is expressed as percentage. ALH of the sperm head is calculated from the amplitude of its lateral deviation about the cell's axis of progression or average path. Methods of measuring sperm motility by CASA are well known in the art, see for example, WO2012061578A2. An increase in sperm motility, as contemplated herein, constitutes an increase in the motility of sperm relative to a suitable control sperm.

vii) In some embodiments sperm with increased motility are provided that are the product of a process comprising incubating sperm one of more steps of the sequential procedure to potentiate the sperm. In some embodiments, the increase in sperm motility can be more than about 10%, 20%, 30%, 40%, 50%, 60%, 70%, 80%, 90%, or 99% relative to a suitable control sperm. In some embodiments, the increase in sperm motility can be at least 5%, at least 10%, at least 20%, at least 30%, at least 40%, at least 50%, at least 60%, at least 70%, at least 80%, at least 90%, or at least 95%. In some embodiments, the increase in sperm motility can be by a factor of at least 10, at least 100, at least 1,000, at least 10,000. In some embodiments, the sperm motility can be increased by from 10% to 200%, from 25% to 150%, from 50% to 100%, or from 70% to 90%. In some embodiments, the increased sperm function or increased sperm motility can be an increase in percentage of hyperactivated sperm. In some embodiments, the increased sperm function or increased sperm motility can be an increase in percentage of progressive motility sperm. In some embodiments, the increased sperm function or increased sperm motility can be an increase in percentage of the hyperactivated sperm. In some embodiments, the level of hyperactivated sperm, progressive motility sperm, is increased so that hyperactivated sperm, progressive motility sperm, or a combination thereof comprise at least about 5%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0%, 25%, 30%, 35%, 40%, 50% or more of the total sperm in a preparation. An increase in sperm motility is indicative of increased sperm function.

### Sperm Capacitation

i) In some embodiments the increased sperm function comprises an increase in sperm capacitation. “Sperm capacitation” refers to the sperm having the ability to undergo acrosomal exocytosis or to develop hyperactive motility and binding to and penetrating through the zona pellucida of an unfertilized egg. Completion of capacitation is manifested by the ability of sperm to bind to the zona pellucida and to undergo ligand-induced acrosomal exocytosis.

ii) In some embodiments, sperm with increased capacitation are provided that are the product of a process comprising incubating sperm in one of more steps of the sequential procedure to potentiate the sperm. In some embodiments, the increase in sperm capacitation can be more than about 10%, 20%, 30%, 40%, 50%, 60%, 70%, 80%, 90%, or 99% relative to a suitable control sperm. In some embodiments, the increase in sperm capacitation can be at least 5%, at least 10%, at least 20%, at least 30%, at least 40%, at least 50%, at least 60%, at least 70%, at least 80%, at least 90%, or at least 95%. In some embodiments, the increase in sperm capacitation can be by a factor of at least 10, at least 100, at least 1,000, at least 10,000. In some embodiments, the sperm capacitation can be increased by from 10% to 200%, from 25% to 150%, from 50% to 100%, or from 70% to 90%. In some embodiments, the level of sperm capacitation is increased so that capacitated sperm can comprise at least about 5%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0%, 20%, 25%, 30%, 35%, 40%, 50% or more of the total sperm in a preparation. An increase in sperm capacitation is indicative of increased sperm function.

### Fertilizing Ability

In some embodiments, the sperm function comprises ability of the sperm to fertilize an egg. The fertilizing ability of a sperm can be determined, for example, by IVF. IVF is a process of fertilization where an egg is combined with sperm outside the body, in vitro (“in glass”). The process involves monitoring and stimulating a woman's ovulatory process, removing an egg or eggs from the woman's ovaries and letting sperm fertilize them in a culture medium in a laboratory. In an embodiment sperm with increased fertilizing ability are provided that are the product of a process comprising incubating sperm in one of more steps of the sequential procedure to potentiate the sperm. In some embodiments, the increase in fertilizing ability can be more than about 10%, 20%, 30%, 40%, 50%, 60%, 70%, 80%, 90%, or 99% relative to a suitable control sperm. In some embodiments, the increase in fertilizing ability can be at least 5%, at least 10%, at least 20%, at least 30%, at least 40%, at least 50%, at least 60%, at least 70%, at least 80%, at least 90%, or at least 95%. In some embodiments, the increase in fertilizing ability can be by a factor of at least 10, at least 100, at least 1,000, at least 10,000. In some embodiments, the fertilizing ability can be increased by from 10% to 200%, from 25% to 150%, from 50% to 100%, or from 70% to 90%. In some embodiments, the level of fertilizing ability is increased so that the number of sperm able to fertilize an egg is at least about 5%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0%, 20%, 25%, 30%, 35%, 40%, 50% or more of the total sperm in a preparation. An increase in fertilizing ability is indicative of increased sperm function and increased fertilization.

### Generating Embryos

i) In some embodiments, sperm function comprises generating an embryo. In some embodiments, the sperm with increased function prepared by methods herein is provided access to an egg to promote fertilization, wherein promoting fertilization can comprise generation of an embryo. In some embodiments, the sperm with increased function prepared by the methods herein is provided access to an egg in vitro, thereby generating the embryo in vitro. In some embodiments, the sperm with increased function prepared by the methods disclosed herein is provided access to an egg in vivo by IUI of the sperm, thereby generating the embryo in vivo. In some embodiments, the sperm which has been incubated under one or more steps of the sequential procedure to potentiate the sperm is inseminated in the reproductive tract of a female subject and providing access to an egg to generate an embryo occurs in vivo. In some embodiments, where the embryo is generated in vitro, the embryo can be cryopreserved for later use or can be further cultured in vitro to enable embryonic development. In some embodiments, the embryo is developed to at least a 2-cell stage prior to cryopreserving and/or implantation into a female subject. In some embodiments, the embryo is developed to a developmental stage greater than the 2-cell stage in vitro prior to further processing. In some embodiments, the embryo is developed to a blastocyst stage in vitro prior to further processing (e.g., cryopreservation or implantation into a female subject to develop into a full offspring). For in vitro incubation and culture of embryos during via ART procedures, a range of suitable media are available, the types and compositions of which are well known to those of skill in the art. Preferably the culture medium contains at least water, salts, nutrients, essential amino acids, vitamins and hormones, and may also include one or more growth factors. A variety of suitable culture media is commercially available, for example Earle's media, Ham's F10 media and human tubal fluid (HTF) media. The present disclosure also contemplates the co-culture in vitro of embryos on a layer of ‘feeder cells’ by methods known in the art. Appropriate ‘feeder cells’ for co-culture may include, for example, bovine oviductal cells or human tubal epithelial cells.

ii) Those of skill in the art will appreciate that the advantages offered by the sperm with increased function prepared by the methods disclosed herein are not limited to increasing fertilization. Rather, the methods and preparation of the present invention are equally applicable as treatment to promote fertilization, whether the embryos are produced in vitro via ART or in the reproductive tract of the animal. The methods of the present invention are applicable to improving fertilization, embryo viability, embryo implantation and pregnancy rates in assisted or otherwise unassisted pregnancies. Embodiments of the present disclosure also provide for methods of increasing the fertilizing ability of sperm in male animals.

iii) In the context of this specification, the terms “embryo with increased viability” and “embryo with longer viability” mean an increase or enhancement in the likelihood of survival of an embryo(s) which has been generated by the mammalian sperm of the methods and preparation disclosed herein, for example, a mammalian sperm with one or more increased sperm function, compared to the likelihood of survival of an embryo(s) which has been generated by a suitable control sperm. In some embodiments, the embryo is generated by an ART e.g., IVF or ICSI. In some embodiments, the embryo is generated in vivo in the reproductive tract of a female mammalian subject by artificial insemination (AI).

iv) For the purposes of the present disclosure, embryo viability may be reflected in a number of indicators. For example, increased embryo viability may result in increased embryo implantation rates following fertilization, decreased pre- and post-implantation embryo lethality, increased clinical pregnancy rates or increased birth rates. The present disclosure therefore also relates to methods of preventing apoptosis or retarded development in embryos and to methods of increasing pregnancy rates in animals. The embryo viability can refer to viability of an embryo in vitro or in vivo.

v) In some embodiments, sperm with ability to generate an embryo with increased viability is provided that are the product of a process comprising incubating sperm one of more steps of the sequential procedure to potentiate the sperm. In some embodiments, providing the sperm with increased function access to an egg promotes fertilization. In some embodiments, promoting fertilization comprises generation of an embryo(s) with increased viability. In some embodiments, the increase in viability of embryo generated by the sperm prepared by methods herein upon access to an egg can be more than about 10%, 20%, 30%, 40%, 50%, 60%, 70%, 80%, 90%, or 99% relative to an embryo generated by a suitable control sperm. In some embodiments, the increase in embryo viability can be at least 5%, at least 10%, at least 20%, at least 30%, at least 40%, at least 50%, at least 60%, at least 70%, at least 80%, at least 90%, or at least 95%. In some embodiments, the increase in embryo viability can be by a factor of at least 10, at least 100, at least 1,000, at least 10,000. In some embodiments, the embryo viability can be increased by from 10% to 200%, from 25% to 150%, from 50% to 100%, or from 70% to 90%. In some embodiments, the level of sperm that can generate an embryo with increased viability is at least about 5%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0%, 20%, 25%, 30%, 35%, 40%, 50% or more of the total sperm in a preparation. Generation of an embryo with increased viability is indicative of increased sperm function and/or increased fertilization.

vi) Typically, the cleavage stage of embryo occurs during the first three days of culture. The in vitro generated embryo can be cryopreserved and transferred at a later stage, or immediately transferred to a female subject by embryo transfer. “Embryo transfer” is the procedure in which one or more embryos and/or blastocysts are placed into the uterus or fallopian tubes. In the traditional IVF process, embryos are transferred to the uterine cavity two days after fertilization when each embryo is at the four (4) cell stage or three days after fertilization when the embryo is at the eight (8) cell stage. It has been recognized that it may be desirable to use embryos at the blastocyst stage when reached at day five to seven of culture. The present disclosure allows for embryo transfer at any time along the spectrum of embryo/blastocyst development. Through visual observation, such as by with the use of microscopy, blastocysts or embryos are considered ready to be transferred to the uterus when the blastocele is clearly evident and comprises greater than 50% of the volume of the embryo. In an in vivo environment, this stage would normally be achieved four to five days after fertilization, soon after the embryo has traversed the fallopian tube and arrives in the uterus. Embryonic developmental stage can be determined by visual observation of the embryo using microscopy, the embryo will display certain determined physical or morphological features simultaneously before it is implanted into the uterus. The state of blastocyst maturity will be determined to be the range II AB-VI AA according to classification of Gardner et al (1998).

vii) The methods disclosed herein result in generation of embryos with increased rate of progressing to 2-cell developmental stage, blastocyst developmental stage, or development to an offspring and live birth. In some embodiments, sperm which can generate an embryo with ability to develop through normal developmental stages (e.g., 2-cell stage, blastocyst stage, development into an offspring and live birth) is provided that are the product of a process comprising incubating sperm one of more steps of the sequential procedure to potentiate the sperm. In some embodiments, providing the sperm with increased function access to an egg promotes fertilization. In some embodiments, promoting fertilization comprises generation of embryos with increased ability to develop through normal developmental stages (e.g., 2-cell stage, blastocyst stage, development into an offspring and live birth). In some embodiments, increase in rate of an embryo progressing through normal developmental stages, generated by the sperm prepared by methods can be more than about 10%, 20%, 30%, 40%, 50%, 60%, 70%, 80%, 90%, or 99% relative to an embryo generated by suitable control sperm. In some embodiments, the increase in rate of an embryo progressing through normal developmental stages can be at least 5%, at least 10%, at least 20%, at least 30%, at least 40%, at least 50%, at least 60%, at least 70%, at least 80%, at least 90%, or at least 95%. In some embodiments, the increase in rate of an embryo progressing through normal developmental stages can be by a factor of at least 10, at least 100, at least 1,000, at least 10,000. In some embodiments, the rate of embryo progressing through normal developmental stages can be increased by from 10% to 200%, from 25% to 150%, from 50% to 100%, or from 70% to 90%. In some embodiments, the level of sperm that can generate an embryo with ability to progress through normal developmental stages is at least about 5%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0%, 20%, 25%, 30%, 35%, 40%, 50% or more of the total sperm in a preparation. Generation of an embryo with ability to progress through one or more normal developmental stages is indicative of increased sperm function and/or increased fertilization.

viii) In vivo, an embryo attaches or implants to a wall of the uterus, creates a placenta, and develops into a fetal offspring during gestation until childbirth. Testing to determine whether one or more embryos have implanted into the endometrium, i.e, whether the procedure has resulted in successful pregnancy inception, is performed two weeks after transfer using blood tests on β-hCG (human chorionic gonadotropin), for example, and other techniques commonly known in the art. U.S. Pat. No. 4,315,908A sets forth a method for detecting β-hCG in the urine by radioimmunoassay. U.S. Pat. No. 8,163,508B2 provides a method and a kit for predicting pregnancy in a subject by β-hCG method by determining the amount of an early pregnancy associated isoform of β-hCG in a sample. Such methods of diagnosis and others are useful within the scope of the disclosure.

ix) In some embodiments, sperm with ability to generate an embryo with improved implantation rate or improved rate of pregnancy is provided that are the product of a process comprising incubating sperm one of more steps of the sequential procedure to potentiate the sperm. In some embodiments, providing the sperm with increased function access to an egg promotes fertilization. In some embodiments, promoting fertilization comprises generation of an embryo with improved implantation rate or improved rate of pregnancy. In some embodiments, the increase in implantation rate of an embryo generated by the sperm prepared by methods herein or pregnancy rate upon implantation of an embryo can be more than about 10%, 20%, 30%, 40%, 50%, 60%, 70%, 80%, 90%, or 99% relative to an embryo generated by a suitable control sperm. In some embodiments, the increase in an embryo implantation rate or pregnancy rate can be at least 5%, at least 10%, at least 20%, at least 30%, at least 40%, at least 50%, at least 60%, at least 70%, at least 80%, at least 90%, or at least 95%. In some embodiments, the increase in rate of embryo implantation or rate of pregnancy can be by a factor of at least 10, at least 100, at least 1,000, at least 10,000. In some embodiments, the embryo implantation or pregnancy rate can be increased by from 10% to 200%, from 25% to 150%, from 50% to 100%, or from 70% to 90%. In some embodiments, the level of sperm that can generate an embryo with increased implantation rate or improved pregnancy rate is at least about 5%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0%, 20%, 25%, 30%, 35%, 40%, 50% more of the total sperm in a preparation. Generation of embryos with improved implantation (i.e., increased rate of implantation) or increased pregnancy rate upon implantation is indicative of increased sperm function and/or increased fertilization.

### Mammalian Sperm

i) The methods disclosed herein comprise increasing one or more sperm functions to promote fertilization. Preparations of sperm with increased function are also provided. As described herein, the sperm can be from a vertebrate, preferably a mammal.

ii) Without limitation, mammals include humans, rodents, primates, wild or domesticated animals, including feral animals, farm animals, sport animals, and pets. Rodents include, for example, mice, rats, and hamsters. Domestic and game animals include, for example, cows, horses, pigs, deer, bison, buffalo, feline species, (e.g., domestic cat), and canine species, (e.g., dog, fox, wolf), avian species, (e.g., chicken, emu, ostrich), and fish, (e.g., trout, catfish and salmon). The mammalian sperm can be from a non-human mammal including, an ungulate, such as an even-toed ungulate (e.g., pigs, peccaries, hippopotamuses, camels, llamas, chevrotains (mouse deer), deer, giraffes, pronghorn, antelopes, goat-antelopes (which include sheep, goats and others), or cattle) or an odd-toed ungulate (e.g., horse, tapirs, and rhinoceroses), a non-human primate (e.g., a monkey, chimpanzees, cynomologous monkeys, spider monkeys, and macaques, e.g., Rhesus.), a Canidae (e.g., a dog) or a cat. The mammalian sperm can be from a member of the Laurasiatheria superorder. The Laurasiatheria superorder can include a group of mammals as described in Waddell et al (1999). The Members of the Laurasiatheria superorder can include Eulipotyphla (hedgehogs, shrews, and moles), Perissodactyla (rhinoceroses, horses, and tapirs), Carnivora (carnivores), Cetartiodactyla (artiodactyls and cetaceans), Chiroptera (bats), and Pholidota (pangolins). A member of Laurasiatheria superorder can be an ungulate, e.g., an odd-toed ungulate or even-toed ungulate. The mammalian sperm can be from a member of Carnivora, such as a cat, or a dog. In some embodiments, the mammalian sperm is a human, non-human primate, porcine, bovine, equine, ovine, canine, feline, or murine sperm. In some embodiments, the mammalian sperm is a human sperm.

iii) In some embodiments, the mammalian sperm is provided by a healthy male mammal. In some embodiments, the mammalian sperm is from a male with some sperm dysfunction as DNA mutations or low sperm count, reduced motility of sperm, and abnormal morphology of sperm. In some embodiments, the mammalian sperm can be from a subfertile male or an oligospermic male. The mammalian sperm can be from a male suffering from, for example, Oligospermia, Teratospermia, Asthenoospermia, or Oligoasthenoteratospermia. Oligospermia refers to a condition characterized by sperm concentration of <20 million/mL. Asthenospermia refers to a condition characterized by reduced sperm motility. Teratospermia refers to a condition characterized by presence of sperm with abnormal morphology. Oligoasthenoteratospermia refers to a condition that includes Oligospermia (low number of sperm), Asthenospermia (poor sperm movement), and Teratospermia (abnormal sperm shape). In some embodiments, the sperm is obtained from a subfertile male or an oligospermic male, e.g., having a sperm count below about: 20, 19, 18, 18, 16, 15, 14, 13, 12, 11, 10, 9, 8, 7, 6, 5, 4, 3, 2 million/mL, e.g., less than 15 million/mL.

iv) In some embodiments, the sperm are separated (or isolated) from semen prior using the method described herein. Any method of sperm enrichment or isolation can be used in combination with the invention, including density gradient centrifugation, swim-up, microfluidics, or a combination thereof.

v) In some embodiments, sperm used in the methods provided by the invention are either fresh or from a preserved stock. For example, sperm are recovered from cryogenic storage prior to treatment. In other embodiments, prior to treatment, the sperm are recovered from non-cryogenic storage.

vi) In some embodiments, different quantities of sperm can be used in the methods provided by the invention, including fractions of a single ejaculate or a whole ejaculate. In some embodiments, the sperm are pooled from at least two ejaculates (e.g., 2, 3, 4, 5, 6, or more ejaculates).

### Methods for Obtaining Sperm Samples

i) Several methods of viable sperm collection such as masturbation into containers, the gloved-hand method, use of an artificial vagina, and electro-ejaculation. Animal semen can be collected by using artificial vagina, electro-ejaculator, or by massaging the ampule of the animal by hand. It can also be directly collected from any section of the male reproductive tract including testicular sperm, and sperm obtained from caput, corpus or cauda epididymis using different methodologies such as puncture of the testis or epididymis using surgical procedures or removing the testis or epididymis and collecting the sperm in surrounding media.

ii) In some embodiments, sperm used in the methods provided by the invention may be freshly collected sample from a source animal (e.g., a mammal), or can be previously thawed or cryopreserved sample. At the time of collection, or subsequently, the collected sperm may be combined with any of a number of various buffers that are compatible with sperm, such as trichloroacetic acid (TCA), 4-2(2-hydroxyethyl)-1-piperazineethanesulfonic acid (HEPES) or phosphate-buffered saline (PBS). The collected sperm suspension can also contain a variety of other additives to keep sperm alive. Exemplary additives include protein sources, antibiotics, growth factors, and compositions that regulate oxidation/reduction reactions intracellularly and/or extracellularly. Alternatively, semen can be collected into an empty container and then subsequently contacted with a buffer within several minutes to hours after collection to form the sperm suspension. In some embodiments, the sperm cells can be collected directly into a container with medium (e.g., M199, Synthetic Oviduct Fluid, PBS, BO, Test-yolk, Tyrode's, HBSS, Ham's F10, HTF, Menezo's B2, Menezo's B3, Ham's F12, DMEM, TALP, Earle's Buffered Salts, CZB, KSOM, BVWV Medium, and emCare Media. In some embodiments, TALP, CZB, etc).

iii) In some embodiments, sperm collection comprises treatment of hyperviscous semen or washing sperm before performing the methods disclosed herein. Washing involves centrifuging a sample of semen or thawed sperm through a diluting wash media, which allows collection of a sperm-rich pellet. Following a wash step, or in place of it, a procedure of isolation of motile sperm from a sample can be done.

iv) In some embodiments, the sperm are isolated from semen prior to use in methods disclosed herein. In some embodiments, sperm with increased function can be further enriched, from sperm prepared according to methods disclosed herein. Generally, sperm are isolated or enriched by allowing the motile sperm to swim away from the dead sperm, non-motile sperm and debris (sperm swim-up), by centrifuging the sperm through a density gradient, or by passing the sperm through a column that binds the dead sperm and debris. Isolating (or enriching) sperm from semen is performed by a method selected from the wash and spin method, the sedimentation method, the direct swim-up method, the pellet and swim-up method, and the buoyant density gradient method. These methods are well known in the art. They are traditionally used in ART and described in detail in Brinsden (1999). In some embodiments, the sperm prepared by the methods disclosed herein can be further enriched for motile sperm by isolation procedures such as the sedimentation method, the direct swim-up method, the pellet and swim-up method, and the buoyant density gradient method.

v) The direct swim-up method implies self-selection of motile sperm, essentially comprising layering an aliquot of medium on top of a semen sample or a preparation of sperm disclosed herein and allowing it to stand at room temperature for at least 30 min. The motile sperm cells migrate into the top layer (medium), from which they can be recovered. The method may also include centrifugation steps.

vi) The method may be varied and combined with further isolation/separation techniques, depending on the number of motile cells in the sample. For example, the swim-up procedure can be performed through the layering of 1 mL of medium containing albumin on 1 mL of underlying seminal liquid in a test tube. After 1 h of incubation at 37° C. in the air or in 5% CO₂ the upper phase of the medium to which the sperm with better motility characteristics have migrated is collected. This technique may also comprise or be combined with a centrifugation step, for example centrifugation on density gradients. The separated, isolated or enriched sperm are then used in methods disclosed herein or can be cryopreserved before being further processed, for example. In case of the preparation of sperm prepared by methods herein, they can be used for IVF, ICSI or AI following enrichment steps or may be cryopreserved for later use, for example. Accordingly, for any of these isolations, or enrichment methods, the sample may be semen, partially purified sperm, purified sperm, or sperm with increased function prepared by methods herein. In some embodiments, the percentage of motile cells is increased by at least 10%, at least 20%, at least 50%, at least 75%, at least 80%, or about 100% after isolating or enriching the sperm using isolation methods, such as direct swim-up, the pellet and swim-up method, and the buoyant density gradient method compared to untreated semen sample or unenriched sperm preparation.

vii) In some embodiments, after isolation, enrichment and washing, the sperm pellet can be resuspended in a medium suitable for further processing, including preservation medium, HTF medium for culturing, medium of the sequential procedure to potentiate the sperm. As it relates to sperm with increased function prepared by methods disclosed herein, the sperm preparation can be resuspended in preservation medium, HTF medium for culturing, medium for insemination, assays of fertilization potential as described herein, IVF, freezing, IUI, cervical cap insemination, and the like. The sperm may be added to medium or the medium can be added to the sperm. The medium can be balanced salt solution which may contain zwitterionic buffers, such as TES, HEPES, PIPES, or other buffers, such as sodium HCO₃⁻. In general, the medium for diluting sperm or culturing sperm, oocytes, embryos or embryonic stem cells is a balanced salt solution, such as M199, Synthetic Oviduct Fluid, PBS, BO, Test-yolk, Tyrode's, HBSS, Ham's F10, HTF, Menezo's B2, Menezo's B3, Ham's F12, DMEM, TALP, Earle's Buffered Salts, CZB, KSOM, BWW Medium, and emCare Media (PETS, Canton, Tex.). In some embodiments, TALP or HTF is used for sperm culture medium, and CZB is used for embryo culture medium. The sperm, or embryo of the present disclosure can be preserved in a cryogenic medium comprising a cryoprotectant.

### Suitable Control Sperm

i) A suitable control sperm can be sperm incubated under control conditions, e.g., in a control buffer such as, HTF medium or modified HTF medium. HTF comprises a sodium HCO₃ buffering system and may be utilized for uses requiring a CO₂ atmosphere during incubation. Modified HTF comprises a combined sodium HCO₃ and HEPES buffer (HTF-HEPES). Suitable examples of HTF medium or modified HTF medium include those that are commercially available from Irvine Scientific, Santa Ana, California. In some embodiments, the HTF medium was used from which sodium HCO₃⁻ has been omitted. The sperm may be incubated for a period sufficient to provide a measurable change in the motility (or other characteristics) of the sperm; in specific embodiments of the method, incubation is from 1 min to 24 h, 15 min to 3 h, 30 min to 1.5 h, about 1 h, or any subrange or subvalue thereof. It is understood that a suitable control sperm can be at least one sperm or a population of sperm, for example, a sperm preparation, or a sperm suspension.

### Sperm Preparation

i) In some embodiments, the invention provides sperm preparations, such as preparations of activated or potentiated sperm. These are collectively “sperm preparations provided by the invention” or “preparations provided by the invention”. In some embodiments the invention provides preparations of hyperactivated sperm comprising at least 5% hyperactivated sperm, e.g., at least about: 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0%, or more hyperactivated sperm, e.g., between about: 5-20, 8.5-20, 10-20, or 12.5-20%. As the skilled artisan will appreciate, sperm may be separated based on HA phenotype, but in some embodiments, the foregoing percentages are based on preparations that have not been activated and then sorted based on HA (however, in some embodiments, sperm preparations may have been pre-processed, e.g., to separate or otherwise enrich sperm from other seminal components, including certain irregular sperm).

ii) In some embodiments, the invention provides a preparation of sperm prepared by any one of the methods provided by the invention.

iii) In some embodiments, the invention provides preparations of sperm prepared by enriching sperm from semen of a male subject, such as a normospermic male, subfertile male, or oligospermic male, e.g., a subfertile (including oligospermic) male, incubating the sperm in one of more steps of the sequential procedure to potentiate the sperm.

iv) For any of the preparations provided by the invention, sperm can be from any male subject, such as a mammal, and in some embodiments, a human. In some embodiments, the human is a normospermic male, or in other embodiments, the male is an oligospermic or subfertile (e.g., low sperm motility) subject.

### Promoting Fertilization

i) The preparation of sperm with increased function prepared by the methods disclosed herein can be useful to promote fertilization. Accordingly, the present disclosure relates to a method of promoting fertilization. The method comprises incubating sperm under one of more steps of the sequential procedure to potentiate the cells and providing the sperm with increased function with access to an egg under conditions to promote fertilization. The preparation of sperm with increased function can be applied in IVF, ICSI, AI (e.g., IUI) in human as well as in the biomedical research industry of animal models for human diseases (infertility, sperm dysfunction), and in the breeding and agricultural industries. The sperm with increased function prepared by the methods disclosed herein can be provided access to an unfertilized egg of the same species as the sperm to promote IVF, ICSI, or can be used for AI, including for example, IUI of female subjects of the same species as the sperm.

### In Vivo Fertilization

i) The sperm with increased function prepared by the methods disclose herein can be useful to promote fertilization in vivo by providing the sperm with increased function access to an egg in the reproductive tract of a female subject of the same species as the sperm. In vivo fertilization can be done by AI of sperm, for example, by intracervical insemination or IUI. Standard AI and IUI, and other methods are well known to those of skill in the art. In some embodiments, the sperm with increased function is provided access to an egg in the reproductive tract of a female subject by IUI of the said sperm to promote fertilization of the egg. In other embodiments, the sperm can access to an egg in vivo by IUI of a mammalian sperm which has been incubated under one of more steps of the sequential procedure to potentiate the sperm. The sperm that is injected may be used as held in suitable liquids. Liquid used for this purpose may be those liquids generally used as a medium for AI.

### In Vitro Fertilization (IVF)

i) The present methods and preparation of sperm disclosed herein is of particular benefit in promoting fertilization by ART, e.g., embryo viability following ART, and in particular IVF. Other suitable ART techniques to which the present disclosure is applicable include, but are not limited to, gamete intrafallopian transfer (GIFT), zygote intrafallopian transfer (ZIFT), blastocyst transfer (BT), ICSI, gamete, embryo and cell cryopreservation, in vitro preparation of embryos for embryo biopsy and other forms of embryo micromanipulation including formation of embryos by nuclear transfer and production transgenic lines and genetically modified lines. It is also applicable to production of embryonic stem cell lines.

ii) In some embodiments, the sperm with increased function prepared by the methods disclosed herein can be used to fertilize an egg in vitro, such as for example, by microinjection, including ICSI, and other methods well known to those in the art. Typically, in IVF, after fertilization, the cells are grown to the blastocyst stage and then implanted. The methods disclosed herein result in increase in formation of an embryo with longer viability and increased ability to develop into a 2-cell stage, blastocyst stage. Accordingly, the preparation of sperm disclosed herein can be useful IVF procedures, including, for example ICSI.

iii) The methods of the present disclosure encompass providing the sperm prepared by methods herein with access to an egg to promote IVF. Providing the sperm access in vitro to the egg may be carried out in an appropriate medium. The medium used for this purpose can be a medium generally used as a medium for IVF, for example, HTF medium. Temperature conditions for providing access may be a general temperature to be used IVF, for example, can be an average body or a temperature close thereto of the mammal. Time for providing access may be any time that is generally required IVF, but not particularly limited, and preferably from 6 to 24 h. IVF rate can be determined by incubating one or more sperm with matured oocytes for about 24 h.

### Fertilization Uses

i) These methods and preparation of sperm disclosed herein are generally applicable to many species, including human, bovine, canine, equine, porcine, ovine, avian, rodent and others. Although useful whenever fertilization is desired, the present methods have particular use in animals and humans that have a fertilization dysfunction in order to increase the likelihood of conception. Such dysfunctions include low sperm count, reduced motility of sperm, and abnormal morphology of sperm. Accordingly, the methods disclosed herein can be useful for preparation of sperm with increased function in infertility clinics prior to their use IVF or IUI. The methods described herein can be used to improve AI, IVF or ICSI in exotic species and/or endangered species. As such the methods can find use for promoting fertilization in animals-maintained captive in a zoo, and in conservation programs aiming to improve reproduction in animals that are close to extinction in the wild. For example, the methods and preparation of sperm of the present disclosure can be used to improve fertilization and pregnancy rates in animal husbandry, for species of agricultural value, and in species bred for conservation purposes.

ii) In addition, the methods and compositions of the present invention are useful in AI procedures, e.g., in commercial breeding. The method can be carried out with sperm from domesticated animals, especially livestock, as well as with sperm from wild animals (e.g., endangered species). For example, as disclosed herein, embodiments of the methods and compositions of the disclosure find application in bovine reproduction. The methods and preparation can be useful for AI in the livestock production industry where it is desirable to influence the outcome towards offspring having one or more preferred characteristics or traits by introducing specific genetically determined traits into the livestock, e.g., offspring of a particular gender, offspring with enhanced milk production, offspring for quality meat production. Use of the methods described herein will result in improved pregnancy rates. Mammalian sperm are frequently damaged by freezing and thawing and results in lower fertility. By improving the performance of the viable sperm, sperm prepared by methods disclosed herein when used for insemination may promote a higher pregnancy rate per estrus cycle, reducing the number of cycles required to ensure conception and hence reducing the overall cost of AI.

iii) Semen from animals with highly desirable traits could be used to inseminate more females because fewer cycles would be needed to ensure conception in any one female. For such applications, the semen is obtained from a male with desired characteristics. In order to influence gender outcome of the resulting offspring, the sperm preparation can be sorted into X- and Y chromosome bearing cells, and/or enriched for sperm with one or more increased sperm function disclosed herein. The sperm may be sorted by commonly used methods, for example, as described in U.S. Pat. No. 5,135,759A, using a flow cytometer/cell sorter into X and Y chromosome-bearing sperm enriched populations. The sperm prepared by the methods disclosed herein can be sorted the into a population comprising a certain percent X chromosome bearing or Y chromosome bearing sperm cells. For example, the sperm of one of the populations may comprise at least about 65% X chromosome bearing or Y chromosome bearing sperm cells, at least about 70% X chromosome bearing or Y chromosome bearing sperm cells, at least about 75% X chromosome bearing or Y chromosome bearing sperm cells, at least about 80% X chromosome bearing or Y chromosome bearing sperm cells, at least about 85% X chromosome bearing or Y chromosome bearing sperm cells, at least about 90% X chromosome bearing or Y chromosome bearing sperm cells, or even at least about 95% X chromosome bearing or Y chromosome bearing sperm cells. In some embodiments, the sorting can be done prior to preparing the sperm with increased function as disclosed herein. In some embodiments, the sorting can be done prior to providing the sperm with increased function with access to an egg for fertilization as in IVF, ICSI or AI.

iv) The methods and preparations provided by the invention can be used in assisted fertilization, such as IVF, including by ICSI. In some embodiments, any of the methods provided by the invention can include the step of providing the sperm to a female reproductive tract. In some embodiments, a sperm preparation provided by the invention (having increased sperm function) can be provided access to an egg for a time sufficient to fertilize the egg, which egg may be ex vivo (e.g., IVF, including ICSI) or, in some embodiments, in a female reproductive tract. Such methods, in some embodiments, entail a subsequent implantation of the fertilized egg in a female carrier.

### EXAMPLES

The present disclosure will be described in greater detail by way of the following specific examples. The following examples are offered for illustrative purposes and are not intended to limit the invention in any manner. Those of skill in the art will readily recognize a variety of non-critical parameters that can be changed or modified to yield alternative embodiments according to the invention.

### Example 1: Materials and Methods

Reagents and mediums: All reagents and chemicals were purchased from Sigma Aldrich (St. Louis, MO) unless specified. Medium used for human sperm incubation was HTF-HEPES containing 101.5 mM NaCl, 4.7 mM KCl, 2.0 mM CaCl2, 0.37 mM KH2PO4, 0.2 mM MgSO4, 0.33 mM sodium pyruvate, 2.77 mM glucose, 21.4 mM sodium lactate, 21 mM HEPES, 10 μg/mL gentamicin, equilibrated at pH 7.3-7.4. Some steps involved addition of 6 mM NaHCO₃ concentration. Alternatively, media were supplemented with either 0.5-1% bovine serum albumin (BSA) or 2-10% human serum substitute supplement (SSS).

For conventional treatment, modified HTF from Irvine Scientific (Santa Ana, CA) supplemented with 10% SSS was employed.

Gamete co-incubation was performed in Irvine Scientific's HTF with 10% SSS under light oil equilibrated with 7.5% CO₂.

Semen Samples: Semen samples were obtained by masturbation into sterile containers from healthy normospermic males or males seeking treatment for infertility. Ejaculates were liquified for up to 2 h at room temperature prior to semen processing and analyzed following WHO standard procedures. Sperm separation from seminal plasma to isolate good-quality viable sperm was performed by density gradient centrifugation or direct swim-up technique.

Density Gradient Centrifugation: Semen samples were centrifuged at 200-500×g for 20 min through 40%/80% discontinuous density gradients (PureSperm; Nidacon, Gothemburg, Sweden). The resulting sperm pellets were resuspended with 2-5 mL of HTF-HEPES medium (either with or without HCO₃) divided into several tubes according to the number of treatments and centrifuged at 500-700×g for 5 min. After removing the supernatant, each pellet was resuspended in the appropriate culture medium adjusting sperm concentration to 5-20 E 6/mL.

Sperm swim-up: Aliquots of 0.5-1 mL of semen were overloaded with 1-2 mL of HTF-HEPES supplemented with 10% SSS and incubated for 1 h at 37° C. The supernatants were then carefully collected, divided into several tubes according to the number of treatments and centrifuged at 400-700×g for 5 min. Each pellet was then resuspended in HTF medium+10% SSS.

Sperm Motility Analysis: Aliquots of 9.7 μl of sperm suspensions were placed on slides under 18 mm×18 mm coverslips, obtaining a preparation depth of 30 μm, and maintained at 37° C. using a temperature-controlled stage. Sperm motility parameters were evaluated using the Sperm Class Analyzer® system (SCA v.6.2.0.1., Microptic SL, Barcelona, Spain), acquiring 60 frames per second. At least 5 microscopic fields and 300 sperm were analyzed. The following parameters were assessed: curvilinear velocity (VCL, μm/s), straight line velocity (VSL, μm/s), average path velocity (VAP, μm/s), linearity (LIN: VSL/VAP×100, %), straightness (STR×100, %), wobble (WOB: VAP/VCL×100, a measure of sperm head side to side movement, %), amplitude of lateral head displacement (ALH, μm) and beat cross frequency (BCF, Hz). Sperm motility was measured and classified as follows: rapid progressive (VCL≥35 μm/s; STR≥80%), medium progressive (VCL≥15 μm/s; STR≥80%), in situ (VCL<15 μm/s; VAP≥5 μm/s) and immotile (VAP<5 μm/s). Percentages of total (rapid progressive+medium progressive+in situ) and progressive (rapid+medium progressive) motility were recorded. Drifting was set in 25 μm/s. Sperm were considered hyperactivated when presenting VCL≥150 μm/s, LIN<50% and ALH≥3.5 μm.

### Example 2: Enhancement of Sperm Motility

This example shows how precise manipulation of sperm incubation conditions, e.g. temperature and intracellular Ca concentration enhances human sperm motility. Semen samples from healthy men were processed by density gradient centrifugation as stated in Example 1.

In the Control treatment sperm were incubated for 2 min with 0.2% DMSO in HTF-HEPES at 37° C. Sperm cells were washed by adding 1 mL of HTF-HEPES medium with 1% BSA following centrifugation at 400×g for 5 min, and the pellet was resuspended in HTF-HEPES medium containing 6 mM HCO₃⁻ and 0.5% BSA. Sperm cells were further incubated for 30 min at 37° C.

In the Temperature treatment sperm were incubated for 2 min with 0.2% DMSO in HTF-HEPES at 37° C. Sperm cells were washed by adding 1 mL of HTF-HEPES medium with 1% BSA following centrifugation at 400×g for 5 min, and the pellet was resuspended in medium containing 6 mM HCO₃⁻ and 0.5% BSA. Sperm cells were further incubated for 30 min at 40° C.

In the Ionophore treatment sperm were incubated for 2 min with 5 μM 4-Br-A23187 (0.2% DMSO) in HTF-HEPES at 37° C. Sperm cells were washed by adding 1 mL of HTF-HEPES medium with 1% BSA following centrifugation at 400×g for 5 min, and the pellet was resuspended in medium containing 6 mM HCO₃⁻ and 0.5% BSA. Sperm cells were further incubated for 30 min at 37° C.

In the Combined treatment sperm were incubated for 2 min with 5 μM 4-Br-A23187 (0.2% DMSO) in HTF-HEPES at 37° C. Sperm cells were washed by adding 1 mL of HTF-HEPES medium with 1% BSA following centrifugation at 400×g for 5 min, and the pellet was resuspended in medium containing 6 mM HCO₃⁻ and 0.5% BSA. Sperm cells were further incubated for 30 min at 40° C.

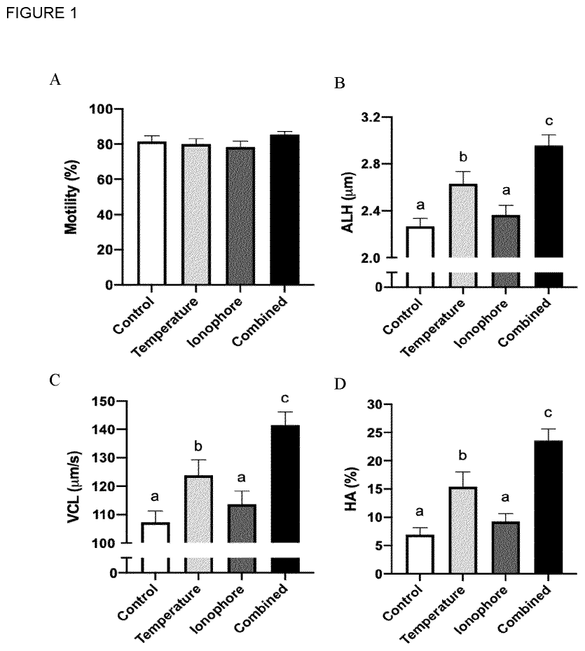

Total motility was not affected for any of the treatments (FIG. 1A). The Combined treatment produced significantly higher values of kinematic parameters associated with HA (e.g., VCL and ALH) compared to the other treatments (FIG. 1B-C). In particular, FIG. 1D shows that 40° C.-treated sperm (Temperature treatment) developed better HA values in comparison to Control procedure (8% HA increase). Similarly, but to a lesser extent, ionophore-treated human sperm also developed higher HA values (3% HA increase). However, a remarkable synergistic effect is observed when sperm are first exposed to ionophore, followed by an incubation at 40° C. (16% HA increase over Control procedure, 50% higher than the 11% that would result from added increases). Values are presented as mean±SEM for 7 independent experiments. Statistical significance of the data was analyzed with one-way ANOVA with the Tukey post-test.

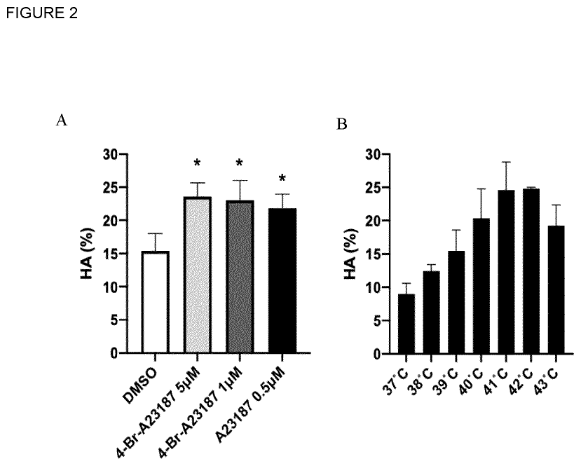

As shown in FIG. 2A the described synergistic effect was observed using different means to pharmacologically increase intracellular Ca2+ concentration temporarily. For example, incubation with either 5 μM 4-Br-A23187 for 2 min, 1 μM 4-Br-A23187 for 5 min or 0.5 μM A23187 for 2 min before incubation at 40° C. for 30 min produced higher levels of HA compared to control.

Moreover, after ionophore pulse different temperatures of 38° C. or more may be used. Incubation of sperm cells for 60 min at temperatures between 38° C. and 43° C. produced an increase in the percentages of HA compared to control (FIG. 2B).

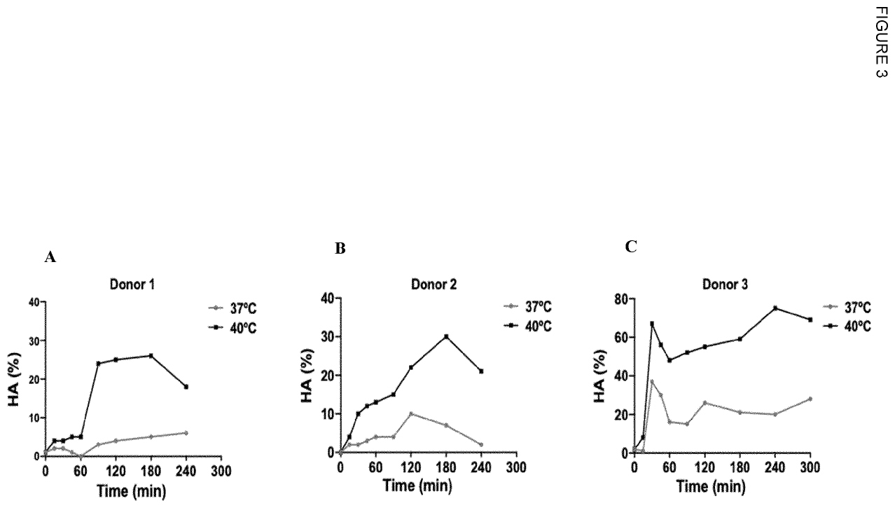

Because of the heterogeneity of human semen, the effect of temperature in the HA increase was variable among donors (FIG. 3A-C). Incubation at 40° C. produced higher levels of HA in comparison to 37° C. at every time point, and in particular during the first hour.

### Example 3: Method for Treating Sperm for Improved Fertilization Rates and Embryo Development on Human IVF

This example describes the use of sperm treated according to certain embodiments of the invention to improve fertility in human subjects undergoing IVF.

Subjects were adult females (i.e., 9 subjects between 18 and 35 years old) undergoing anonymous egg donation. Subjects were treated with standard procedures (e.g., ovulation suppression followed by ovulation stimulation, with hCG triggering injection as indicated) prior to egg retrieval. Fourteen retrieved eggs were divided into two groups: control group, and Combined treatment group. Semen samples of patients attending the reproductive clinic were divided into two halves that were processed by density gradient centrifugation. In the half assigned to the control group (Control treatment), sperm were left 2-4 h at room temperature in HTF supplemented with 10% SSS. For treated sperm (Combined treatment), sperm were kept at room temperature in HTF-HEPES containing 2% SSS for 0.5-2 h at room temperature. Later, sperm were washed with SSS-free medium and incubated for 2 min with 5 μM 4-Br-A23187 in HTF-HEPES at 37° C. Sperm cells were washed by adding 1 mL of HTF-HEPES medium containing 10% SSS and 6 mM HCO₃ following centrifugation at 400×g for 5 min and resuspended in medium containing 6 mM HCO₃⁻ and 10% SSS. Sperm cells were further incubated for 30 min at 37° C. and 60 min at 40° C.

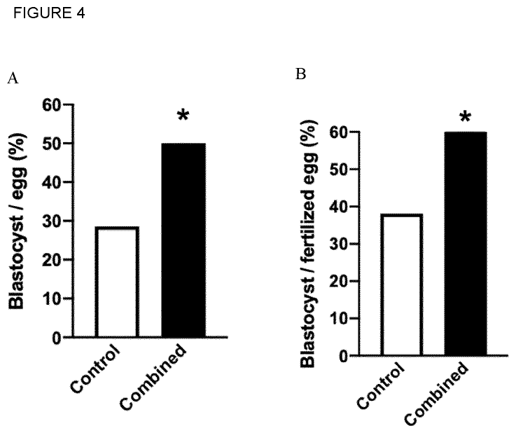

For both the Combined treatment and Control groups, sperm were incubated with eggs in vitro. The number of fertilized eggs (with two pronuclei the following morning) and the number of high-quality blastocysts at 5 and 6 days were recorded (see Table I). High-quality blastocysts were those with score of 3BB or above according to Gardner grading system (Gardner, 2016). The Combined treatment produced a higher number of high-quality blastocysts relative to fertilized eggs (FIG. 4A) and total mature eggs (FIG. 4B) compared to the Control group.

TABLE I
Outcomes of pilot clinical study for the combined treatment.

| | Control | Combined |
|---|---|---|
| # Mature (MII) eggs | 56 | 60 |
| # Fertilized eggs | 42 (75.0) | 47 (78.3) |
| (% over MII eggs) | | |
| # D5 blastocysts | 7 | 14 |
| # D6 blastocysts | 9 | 16 |
| # blastocysts | 16 (28.6) | 30 (50.0) |
| (% over MII eggs) | | |
| # high quality blastocysts | 6 (10.7) | 14 (23.3) |
| according to KIDScore | | |
| algorithm (% over MII eggs) | | |

### Example 4: Time-Lapse Monitoring of Human Embryos

Fertilized eggs were cultured in EmbryoScope® (Vitrolife, Göteborg, Sweden) incubator for time-lapse monitoring for 5-6 days to blastocyst stage. The EmbryoViewer image analysis software (Vitrolife) was employed to establish the time of each developmental event: tPNf, time of pronuclei fading; t2, time at 2-cells; t3, time at 3-cells; t4, time at 4-cells; t5, time at 5-cells; t8, time at 8-cells; tM, time at morula; tB, time at blastocyst. KIDScore™ Day 5 (version 2), developed by EmbryoScope and based on very large multicentric datasets, was employed as a predictive model for embryos' potential for implantation after transfer on day 5 (Gazzo et al, 2020). Compared to the Control group, the Combined treatment produced a higher number of embryos with high potential for implantation according to KIDScore (see Table I).

### FINAL REMARKS

For all numerical bounds describing some parameter in this application, such as “about,” “at least,” “less than,” and “more than,” the description also necessarily encompasses any range bounded by the recited values. Accordingly, for example, the description “at least 1, 2, 3, 4, or 5” also describes, inter alia, the ranges 1-2, 1-3, 1-4, 1-5, 2-3, 2-4, 2-5, 3-4, 3-5, and 4-5, et cetera.

Headings used in this application are for convenience only and do not affect the interpretation of this application.

Preferred features of each of the aspects provided by the invention (e.g., media, compositions, preparations, and methods) are applicable to all the other aspects of the invention mutatis mutandis and, without limitation, are exemplified by the dependent claims and also encompass combinations and permutations of individual features (e.g., elements, including numerical ranges and exemplary embodiments) of particular embodiments and aspects of the invention, including the working examples. For example, particular experimental parameters exemplified in the working examples can be adapted for use in the claimed invention piecemeal without departing from the invention. For example, for materials that are disclosed, while specific reference of each of the various individual and collective combinations and permutations of these compounds may not be explicitly disclosed, each is specifically contemplated and described herein. Thus, if a class of elements A, B, and C are disclosed as well as a class of elements D, E, and F and an example of a combination of elements A-D is disclosed, then, even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-groups of A-E, B-F, and C-E are specifically contemplated and should be considered disclosed from disclosure of A, B, and C; D, E, and F; and the example combination A-D. This concept applies to all aspects of this application, including elements of a composition of matter and steps of method of making or using the compositions.

The foregoing aspects of the invention, as recognized by the person having ordinary skill in the art following the teachings of the specification, can be claimed in any combination or permutation to the extent that they are novel and non-obvious over the prior art-thus, to the extent an element is described in one or more references known to the person having ordinary skill in the art, they may be excluded from the claimed invention by, inter alia, a negative proviso or disclaimer of the feature or combination of features.

### BIBLIOGRAPHY

Alvarez J G, Minaretzis D, Barrett C B, Mortola J F, Thompson I E. The sperm stress test: a novel test that predicts pregnancy in assisted reproductive technologies. Fertil Steril. 1996 February; 65(2):400-5. doi: 10.1016/s0015-0282(16)58107-2.
Brinsden P R, Bourn Hall Clinic. A textbook of in vitro fertilization and assisted reproduction: the Bourn Hall guide to clinical and laboratory practice. The Parthenon Publishing Group. 1999; 564.
Chan P J, Corselli J U, Patton W C, Jacobson J D, King A. Heat-induced hyperactivation. J Assist Reprod Genet. 1998 January; 15(1):32-8. doi: 10.1023/a:1022526305186.
Chan P J, Corselli J U, Patton W C, Jacobson J D, King A. Enhanced fertility after heat-induced hyperactivation. Fertil Steril. 1998 January; 69(1):118-21. doi: 10.1016/s0015-0282(97)00440-8.
Gardner D K, Schoolcraft W B. Culture and transfer of human blastocysts. Curr Opin Obstet Gynecol. 1999 June; 11(3):307-11. doi: 10.1097/00001703-199906000-00013.
Gazzo E, Peña F, Valdéz F, Chung A, Bonomini C, Ascenzo M, Velit M, Escudero E. The KIDScore™ D5 algorithm as an additional tool to morphological assessment and PGT-A in embryo selection: a time-lapse study. JBRA Assist Reprod. 2020 Jan. 30; 24(1):55-60. doi: 10.5935/1518-0557.20190054.
Keppler E L, Chan P J, Patton W C, King A. Aggregation of human sperm at higher temperature is due to hyperactivation. Arch Androl. 1999 January-February; 42(1):35-9. doi: 10.1080/014850199263020.
Küçük T, Sözen E, Buluç B. Effect of heat-induced hypermotility on pregnancy rate in intrauterine insemination for male factor infertility associated with asthenospermia: a prospective, randomized, controlled study. J Assist Reprod Genet. 2008 June; 25(6):235-8. doi: 10.1007/s10815-008-9226-1.
Mann S L, Patton W C, King A, Chan P J. Comparative genomic hybridization analysis of sperm DNA apoptosis after exposure to heat shock. J Assist Reprod Genet. 2002 April; 19(4):195-200. doi: 10.1023/a:1014846113170.
Marin-Briggiler C I, Luque G M, Gervasi M G, Oscoz-Susino N, Sierra J M, Mondillo C, Salicioni A M, Krapf D, Visconti P E, Buffone M G. Human Sperm Remain Motile After a Temporary Energy Restriction but do Not Undergo Capacitation-Related Events. Front Cell Dev Biol. 2021 Nov. 12; 9:777086. doi: 10.3389/fcell.2021.777086.
Navarrete F A, Alvau A, Lee H C, Levin L R, Buck J, Leon P M, Santi C M, Krapf D, Mager J, Fissore R A, Salicioni A M, Darszon A, Visconti P E. Transient exposure to calcium ionophore enables in vitro fertilization in sterile mouse models. Sci Rep. 2016 Sep. 15; 6:33589. doi: 10.1038/srep33589.
Navarrete F A, Aguila L, Martin-Hidalgo D, Tourzani D A, Luque G M, Ardestani G, Garcia-Vazquez F A, Levin L R, Buck J, Darszon A, Buffone M G, Mager J, Fissore R A, Salicioni A M, Gervasi M G, Visconti P E. Transient Sperm Starvation Improves the Outcome of Assisted Reproductive Technologies. Front Cell Dev Biol. 2019 Nov. 5; 7:262. doi: 10.3389/fcell.2019.00262.
Pérez-Cerezales S, Laguna-Barraza R, de Castro A C, Sánchez-Calabuig M J, Cano-Oliva E, de Castro-Pita F J, Montoro-Buils L, Pericuesta E, Fernandez-Gonzalez R, Gutierrez-Adan A. Sperm selection by thermotaxis improves ICSI outcome in mice. Sci Rep. 2018 Feb. 13; 8(1):2902. doi: 10.1038/s41598-018-21335-8.
Sánchez-Cardenas C, Montoya F, Navarrete F A, Hernández-Cruz A, Corkidi G, Visconti P E, Darszon A. Intracellular Ca2+ threshold reversibly switches flagellar beat off and on. Biol Reprod. 2018 Nov. 1; 99(5):1010-1021. doi: 10.1093/biolre/ioy132.
Si Y. Temperature-dependent hyperactivated movement of hamster spermatozoa. Biol Reprod. 1997 December; 57(6):1407-12. doi: 10.1095/biolreprod57.6.1407.
Tateno H, Krapf D, Hino T, Sanchez-Cardenas C, Darszon A, Yanagimachi R, Visconti P E. Ca2+ ionophore A23187 can make mouse spermatozoa capable of fertilizing in vitro without activation of cAMP-dependent phosphorylation pathways. Proc Natl Acad Sci USA. 2013 Nov. 12; 110(46):18543-8. doi: 10.1073/pnas.1317113110.
Waddell P J, Okada N, Hasegawa M. Towards Resolving the Interordinal Relationships of Placental Mammals. Syst Biol. 1999 Mar. 1; 48(1):1-5.
Wiser A, Sachar S, Ghetler Y, Shulman A, Breitbart H. Assessment of sperm hyperactivated motility and acrosome reaction can discriminate the use of spermatozoa for conventional in vitro fertilisation or intracytoplasmic sperm injection: preliminary results. Andrologia. 2014 April; 46(3):313-5. doi: 10.1111/and.12068.
World Health Organization. WHO laboratory manual for the examination and processing of human semen. 5th ed. Geneva, Switzerland: WHO; 2010.

What is claimed is:

1. A method for preparing a mammalian sperm wherein the method comprises:
(a) incubating the mammalian sperm in the presence of Ca²⁺ ionophore
(b) washing the mammalian sperm of step (a) with fresh medium to remove Ca²⁺ ionophore
(c) incubating the mammalian sperm of step (b) at temperature between 38° C. and 43° C. in a medium containing HCO₃ and,
(d) transferring an appropriate concentration of mammalian sperm of step (c) to an appropriate medium,
wherein said mammalian sperm are suitable for use in the assisted reproductive techniques (ART).

2. The method of claim 1, wherein one or more sperm function is selected from the group consisting of: curvilinear velocity, amplitude or lateral head displacement, sperm capacitation, percentage of hyperactivated sperm, and wherein said sperm function is improved relative to a suitable control sperm.

3. The method of claim 1, further comprising transferring the mammalian sperm of step d) in a fertilization medium, a preservation medium, or a culture medium.

4. The method of claim 3, wherein the fertilization medium is any sperm culture media.

5. The method of claim 3, further comprising the step of cryopreserving the mammalian sperm prior to use in the ART.

6. The method of claim 1, wherein the albumin of step a-c) is present or absent.

7. The method of claim 1, wherein the ART comprises IVF of an egg by the mammalian sperm of step (d) to generate an embryo.

8. The method of claim 1, wherein the ART is selected from the group consisting of frozen embryo transfer (FET), IVF, ICSI, gamete intrafallopian tube transfer (GIFT), and zygote intrafallopian tube transfer (ZIFT).

9. The method of claim 1, wherein the ART is AI of the mammalian sperm of step (d).

10. The method of claim 9, wherein said AI is IUI or intracervical insemination

11. The method of claim 1, wherein the mammalian sperm of step (a) is recovered from a cryogenic storage.

12. The method of claim 1, wherein the mammalian sperm of step (a) is recovered from a non-cryogenic storage.

13. The method of claim 1, wherein the mammalian sperm of step (a) is from an normo, oligo, astheno, or teratospermic subject or a subfertile subject.

14. The method of claim 1, wherein the mammalian sperm of step (a) is a human sperm or any mammalian species.

15. The method of claim 1, wherein the mammalian sperm of step (a) is provided as an individual sample or as pool of two or more ejaculates.

16. The method of claim 1, wherein the mammalian sperm of step (a) is enriched from semen prior to step (a) by density gradient centrifugation, swim-up, or microfluidics.
